SEQUENCE LISTING (14 sequences)

SEQ ID NO: 1 - DNA, 16 bp, synthetic construct
  source 1..16: mol_type = other DNA
  modified_base 16: mod_base = OTHER; note = T is biotin labeled
tcctgctcattctcgt

SEQ ID NO: 2 - DNA, 16 bp, synthetic construct
  source 1..16: mol_type = other DNA
  modified_base 13: mod_base = OTHER; note = T is biotin labeled
tcctgctcattctcgt

SEQ ID NO: 3 - DNA, 16 bp, synthetic construct
  source 1..16: mol_type = other DNA
  modified_base 10: mod_base = OTHER; note = T is biotin labeled
tcctgctcattctcgt

SEQ ID NO: 4 - DNA, 16 bp, synthetic construct
  source 1..16: mol_type = other DNA
  modified_base 7: mod_base = OTHER; note = T is biotin labeled
tcctgctcattctcgt

SEQ ID NO: 5 - DNA, 16 bp, synthetic construct
  source 1..16: mol_type = other DNA
  modified_base 4: mod_base = OTHER; note = T is biotin labeled
tcctgctcattctcgt

SEQ ID NO: 6 - DNA, 16 bp, synthetic construct
  source 1..16: mol_type = other DNA
  modified_base 1: mod_base = OTHER; note = T is biotin labeled
tcctgctcattctcgt

SEQ ID NO: 7 - DNA, 16 bp, synthetic construct
  source 1..16: mol_type = other DNA
acgagaatgagcagga

SEQ ID NO: 8 - DNA, 13 bp, synthetic construct
  source 1..13: mol_type = other DNA
agaatgagcagga

SEQ ID NO: 9 - DNA, 10 bp, synthetic construct
  source 1..10: mol_type = other DNA
atgagcagga

SEQ ID NO: 10 - DNA, 16 bp, synthetic construct
  source 1..16: mol_type = other DNA
tggacaaagaggagca

SEQ ID NO: 11 - DNA, 16 bp, synthetic construct
  source 1..16: mol_type = other DNA
tcctgctcattctcgt

SEQ ID NO: 12 - DNA, 16 bp, synthetic construct
  source 1..16: mol_type = other DNA
tgctcctctttgtcca

SEQ ID NO: 13 - DNA, 16 bp, synthetic construct
  source 1..16: mol_type = other DNA
acgagaatgagcagga

SEQ ID NO: 14 - protein, 17 aa, synthetic construct
KGSGSETSGLQEQRNHL

(19) United States
(12) Patent Application Publication
(10) Pub. No.: US 2026/0168953 A1
(43) Pub. Date: Jun. 18, 2026

(54) DENSITY-INDEPENDENT ELECTROCHEMICAL DNA SENSORS THAT USE STERIC HINDRANCE AND REDOX INHIBITION MECHANISMS

(71) Applicant: Valorisation-Recherche Société En Commandite, Quebec (CA)

(72) Inventors: Alexis VALLEE-BELISLE, Outremont (CA); Guichi ZHU, Montréal (CA)

(21) Appl. No.: 18/871,676

(22) PCT Filed: Jun. 16, 2023
(86) PCT No.: PCT/CA2023/050838

§ 371 Date: Dec. 4, 2024

Related U.S. Application Data
(60) Provisional application No. 63/366,476, filed on Jun. 16, 2022.

(51) Int. Cl. G01N 27/327; C12Q 1/6834; G01N 33/543

(52) U.S. Cl. CPC G01N 27/3276; C12Q 1/6834; G01N 27/3277; G01N 33/5438

(57) ABSTRACT

In is provided an electrochemical steric hindrance hybridization assay system comprising a plurality of capturing DNA molecules, a substrate associated with the plurality of capturing DNA molecules; and a plurality of signaling DNA molecules having a core nucleic acid sequence which is complementary to a region of the capturing DNA molecules, has a moiety for binding an analyte entity in close proximity with a reporter moiety, and is configured such that there is an inhibition of the hybridization of the plurality of signaling DNA molecules and reporter activity on the surface associated with the plurality of capturing DNA molecules upon binding of the moiety to the analyte, wherein binding of the analyte to the signaling DNA molecule in proximity to the reporter moiety produces a steric hindrance between each analyte, the analyte and the capturing DNA molecule, the analyte and the substrate; as well as an inhibition of the redox activity due to the interaction with the target.

### CROSS-REFERENCE TO RELATED APPLICATION

The present application is claiming priority from U.S. Provisional Application No. 63/366,476 filed Jun. 16, 2022, the content of which is hereby incorporated by reference in its entirety.

### TECHNICAL FIELD

It is provided an electrochemical steric hindrance hybridization assay for detecting analytes in a sample.

### BACKGROUND

Over the past few decades, electrochemical DNA-based sensors (eDNA sensors) have attracted increasing attention due to their high sensitivity, specificity, low cost, and ability to detect broad classes of biomarkers. The remarkable universality of eDNA sensors relies on their ability to employ any type of recognition elements and signaling mechanism. For example, eDNA sensors have been designed to utilize nucleic acids, small molecules, peptides, proteins and even enzymes as their recognition elements, eDNA sensors have also been engineered using numerous signaling mechanisms, including structure-switching, diffusion-collision, enzyme catalysis, and steric-hindrance. These sensing mechanisms display specific advantages depending on the class of biorecognition element employed, but most of them also display important limitations. Their performance, for example, enormously varies with factors affecting its surface density such as fabrication variation or sensor aging and their electrochemical baseline typical drifts when deployed in the complex sample such as blood.

Few studies have focused on the reproducibility of sensor fabrication and the optimization of their stability over time. While these challenges trigger less academic interest, on the other hand, they continue to significantly limit the commercialization process of eDNA sensors even 20 years after their first development. Various strategies have been proposed to reduce the impact of these limitations. Although these methods have improved the accuracy and precision of eDNA sensors, they remain complex and of limited use to account for all factors affecting sensor reproducibility, such as variation in sensor density linked to fabrication and aging.

There is thus still a need to be provided with more stable and improved eDNA sensors.

### SUMMARY

In accordance to an embodiment, it is provided a system for detecting a target analyte in a sample, the system comprising a plurality of capturing DNA molecules having a first end and a second free end; a first substrate having a surface associated with, at a plurality of locations, each of the first end of the plurality of capturing DNA molecules; and a plurality of signaling DNA molecules, wherein each of the signaling DNA molecule has a core nucleic acid sequence which is substantially complementary to a region of each of the capturing DNA molecule and is capable of hybridizing with the capturing DNA molecule; has a first end being associated with a moiety for binding an analyte entity in close proximity with a reporter moiety; has a second end; and is configured such that there is an inhibition of the hybridization of the plurality of signaling DNA molecules and reporter activity on the surface associated with the plurality of capturing DNA molecules upon binding of the moiety to the analyte; wherein binding of the analyte to the signaling DNA molecule in proximity to the reporter moiety produces a steric hindrance between each analyte; the analyte and the capturing DNA molecule; and the analyte and the substrate.

In an embodiment, the moiety for binding the analyte entity is at a distance of at most 16 nucleotides form the reporter moiety; at a distance of at most 13 nucleotides form the reporter moiety; at a distance of at most 10 nucleotides form the reporter moiety; at a distance of at most 7 nucleotides form the reporter moiety; at a distance of at most 4 nucleotides form the reporter moiety; or preferably at a distance of at most 1 nucleotide form the reporter moiety.

In a further embodiment, the moiety is a at distance of at most 16 nucleotides from the first end of the signaling DNA molecule; at distance of at most 13 nucleotides from the first end of the signaling DNA molecule; at distance of at most 10 nucleotides from the first end of the signaling DNA molecule; at distance of at most 7 nucleotides from the first end of the signaling DNA molecule; at distance of at most 4 nucleotides from the first end of the signaling DNA molecule; or preferably at distance of at most 1 nucleotide from the first end of the signaling DNA molecule.

In another embodiment, the capturing DNA molecule is of at most 16 nucleotides in length; at most 13 nucleotides in length; preferably at most 10 nucleotides in length, or at most 8 nucleotides in length.

In an embodiment, the target analyte is a macromolecule or an antibody.

In a further embodiment, the system described herein is for detecting a small molecule or a polypeptide, wherein the small molecule or the polypeptide competes for the binding of the analyte to the signaling DNA molecule.

In an embodiment, the small molecule is a drug substance, e.g. cocaine.

In a further embodiment, the moiety for binding the analyte is an antigen.

In a supplemental embodiment, the reporter moiety is a redox-reporter or a fluorophore.

In an embodiment, the redox-reporter is methylene blue.

In another embodiment, the substrate is a metallic electrode, a 96-well plate, or a tube.

In an embodiment, the metallic electrode is a gold electrode or a carbon electrode.

In a further embodiment, the substrate is a glass tubing Ag/AgCl reference electrode, a platinum wire counter electrode, or a CTI electrode.

In an embodiment, the substrate is an electrode comprising a gold working electrode (WE), a platinum reference electrode (RE), and a platinum counter electrode (CE).

In another embodiment, the sample is a biological sample from a subject.

In another embodiment, the sample is water, an environment element or food.

In a supplemental embodiment, the biological sample is whole blood, saliva, or urine.

In a particular embodiment, the subject is a human or an animal.

It is also provided a comprising the system as defined herein in a container.

In an embodiment, the container is an Eppendorf with a lid. In another embodiment, the lid contains the capturing DNA molecules.

In a further embodiment, the kit also comprises a potentiostat.

It is further provided a method for the detection of a target analyte in a sample, the method comprising providing the sample suspected of having the target analyte; providing the system as described herein; providing or determining a control amount of the plurality of the capturing DNA molecules having hybridized with the plurality of the signaling DNA molecules in the system in the absence of the target analyte; contacting the sample with the system; determining a test amount of the plurality of capturing DNA molecules having hybridized with the plurality signaling DNA molecules in the system in the presence of the sample; and characterizing the sample has having the target analyte if it is determined that the test amount is lower than the control amount and as lacking the target analyte if it is determined that the test amount is equal to or higher than the control amount.

In an embodiment, the method described herein further comprises quantifying the concentration of the target analyte in the sample based on the comparison of the control amount and the test amount.

In another embodiment, the method described herein further comprises determining the control amount by contacting a control sample known to lack the target analyte with the system as described herein and determining the amount of the plurality of capturing DNA molecule having hybridized with the plurality of signaling DNA molecule.

In an embodiment, the method described herein further comprises determining the test amount and/or the control amount electrochemically.

### BRIEF DESCRIPTION OF THE DRAWINGS

Reference will now be made to the accompanying drawings.

FIG. 1a illustrates the classic electrochemical steric hindrance hybridization assay (eSHHA), the hybridization efficiency of a redox-labeled signaling DNA is reduced through steric hindrance between the large analyte proteins. FIG. 1b illustrates an electrochemical steric hindrance and redox inhibition assay (eSHRI) wherein the location of the recognition element is strategically positioned closer to the gold surface and the redox molecule, resulting in a new sensing mechanism that could take place between the analyte and capturing DNA layer or the gold surface.

FIG. 2a illustrates hybridization efficiencies between a signaling DNA and a complementary capturing DNA attached to an electrode surface drastically decrease when the analyte binds near the electrode surface and the redox molecule. FIG. 2b illustrates that hybridization efficiencies of signaling DNAs are labeled with methylene blue (●, at 5′ end) and biotin (u) at different positions in the strand. The signaling DNA is named according to the distance between biotin and methylene blue. For example, signaling DNA-16 indicates that there are 16 nucleotides between the biotin and methylene blue. Square wave voltammograms (SWV) after 5 mins of hybridization in the absence and presence of streptavidin. In the absence of streptavidin, all signaling DNAs display similar currents while the current decreases significantly in the presence of 100 nM streptavidin as the biotin moves closer to the gold surface and methylene blue.

FIG. 3 illustrates the hybridization efficiency and kinetics of a signaling DNA to a complementary capturing DNA located on the surface of the sensor decrease linearly with decreasing distance between analyte binding site and the electrode surface or the redox molecule, showing in FIG. 3(a) The kinetic profile of surface hybridization of six signaling DNAs and capturing DNA in the absence and presence of 100 nM streptavidin; FIG. 3(b) raw current after 5 mins; FIG. 3(c) signal gain after 5 mins, the signal gain is calculated by the percentage of current decrease with streptavidin versus no streptavidin (gain: Cₛₜᵣₑₚ−C₀/C₀); FIG. 3(d) rate constant with or without streptavidin; and FIG. 3(e) signal gain of the sensor versus time.

FIGS. 4(a) to (f) illustrate the raw current of different surface densities at 30 mins. 300 nM, 100 nM, 50 nM, and 25 nM of capturing DNA concentration were used to create electrodes with different surface densities. The length of capturing DNA used in this assay was 16 nucleotides. The error bars show the standard deviation of the current or signal gain obtained from three experiments.

FIG. 5 illustrates that decreasing the distance between the analyte binding site on the signaling DNA and the electrode surface, and the redox molecule renders the assay insensitive to capturing DNA density. 300 nM, 100 nM, 50 nM, and 25 nM of capturing DNA concentration were used to create electrodes with different surface densities. The signal gain is calculated by the percentage of current decrease with streptavidin versus no streptavidin at 30 mins. The length of capturing DNA used in this assay was 16 nucleotides (nt). The error bars show the standard deviation of the signal gain obtained from three experiments.

FIGS. 6(a) to (f) illustrate the raw current of different capturing DNA length at 30 mins. Capturing DNA with the length of 16, 13, and 10 nucleotides (nt) were employed to vary the hybridization affinity on the surface. The error bars show the standard deviation of the current or signal gain obtained from three experiments.

FIG. 7 illustrates that decreasing the distance between the analyte binding site on the signaling DNA and the electrode surface, and the redox molecule renders the assay insensitive to variation in hybridization affinity between the DNA strands. Capturing DNA with the length of 16, 13, and 10 nucleotides (nt) are employed to vary the hybridization affinity on the surface. The signal gain is calculated by the percentage of current decrease with streptavidin versus no streptavidin at 30 mins. The error bars show the standard deviation of the signal gain obtained from three experiments.

FIG. 8 illustrates protein-induced fluorescence enhancement of methylene blue supports the contact redox-inhibition mechanisms. The fluorescence of the methylene blue-labelled signaling DNA is slightly reduced (˜32% on average) upon binding to its complementary strand (green curve). The fluorescence of the methylene blue-labelled signaling DNA is significantly enhanced upon streptavidin binding within three nucleotides of the methylene blue (<4 nm, red curve), which also creates significant contact redox inhibition activity. The concentration of signaling DNAs and streptavidin is 100 nM, respectively.

FIG. 9 illustrates that the highly sensitive eSHRI remains unaffected by variation in sensor fabrication and factors affecting DNA hybridization (e.g., temperature, matrix buffer), showing in FIG. 9(a) optimal conditions were realized at high surface density (300 nM capturing DNA) using 100 nM of signaling DNA-16 (K_{D}=19 nM) and signaling DNA-01 (K_{D}=15 nM), for classical eSHHA and eSHRI, respectively; in FIG. 9(b) poor surface density employed 25 nM capturing DNA; in FIG. 9(c) weak hybridization affinity employed 10-nt capturing DNA at high density. The error bars show the standard deviation of the signal gain obtained from three experiments.

FIG. 10(a) illustrates the synthesis protocol of THC azide. FIG. 10(b) shows the attachment of THC-azide and methylene blue to the dual labeled DNA (NH₂ and DBCO). FIG. 10(c) illustrates the synthesis protocol of cocaine azide. FIG. 10(d) shows the attachment of a peptide-azide to the dual-labeled DNA (MB and DBCO).

FIG. 11(a) illustrates the kinetic profile of THC antibody assay in buffer using CTI (Conductive Technologies) electrodes, and FIG. 11(b) shows the signal gain of the sensor versus time.

FIG. 12 illustrates eSHRI can also be adapted into a competition format to detect antigens or small molecules.

FIG. 13(a) illustrates THC detection in buffer using a competitive eSHRI assay. FIG. 13(b) shows signal gain of the THC sensor versus time (buffer). FIG. 13(c) illustrates THC detection in saliva using the competitive eSHRI assay. FIG. 13(d) shows signal gain of the THC sensor versus THC concentration in human saliva. In this assay, the free THC molecules was pre-incubated with THC antibodies for 5 mins before spiking signaling DNA into the buffer or saliva. FIG. 13(e) compares THC detection (100 nM) in buffer using the THC sensor (square) with cocaine detection in buffer using the cocaine sensor (circles) using the competitive eSHRI assay.

FIG. 14 illustrates an adaptation of eSHRI in a point-of-care format for rapid detection in a drop of blood. FIG. 14(a) shows the classical three electrodes system employed herein uses a rod gold electrode (diameter=2 mm), a glass tubing Ag/AgCl reference electrode, and a platinum wire counter electrode. FIG. 14(b) shows a small integrated electrode from Micrux Technologies contains a small gold working electrode (WE: 1 mm), a platinum reference electrode (RE), and a platinum counter electrode (CE). FIG. 14(c) illustrates the kinetic profile of streptavidin assay in buffer using Micrux electrodes. FIG. 14(d) shows the kinetic profile of streptavidin assay directly in 5 μL blood using Micrux electrodes. 100 nM signaling DNA-01 and 100 nM streptavidin were used in this assay. The error bars show the standard deviation of the current or signal gain obtained from three experiments.

FIG. 15a illustrates a control strategy consisting in employing an extra “control” electrode with an hybridization assay that contains another antigen/antibody couple that will not interact with molecules present in clinical samples. Different DNA colours represent different DNA sequences.

FIG. 15b illustrates a control strategy consisting in employing an extra “control” electrode with an hybridization assay that contains a antigen-labelled signaling DNA but without antibody. This antigen shall not interact with molecules in the biological sample.

FIG. 15c illustrates a control strategy consisting in employing an extra electrode with an hybridization assay that does not contains a recognition element on the signaling strand.

FIG. 15d illustrates a control strategy which employs an extra signaling and capturing DNA strands with a different redox element that can also hybridize on the same electrode.

FIG. 15e illustrates a variation of the control electrode proposed in the strategy as illustrated in FIG. 15c.

FIG. 16 illustrates a potential workflow of the THC assay where the THC contained in a swab that had been used to brush someone's gum is released into a solution containing the THC antibody. Following a short incubation, the lid of the Eppendorf, which contains the dried signalling DNA (THC-DNA and control-DNA, here cocaine-DNA), is closed and the solution is mixed for 10 sec to solubilize the signalling DNA. This solution is then added to the electrodes (THC and cocaine electrodes) for analysis (see FIG. 15b for control strategy employed here). Here the analysis is performed on a portable potensiostat controlled by an app via Bluetooth. In absence of THC, both electrodes detect similar amount of signalling DNA. In presence of THC, less THC antibodies are available to bind to the THC-DNA and more THC-DNA can bind the THC electrode, leading to an increase in electrochemical signal on the THC electrode.

FIG. 17 illustrates hybridization kinetics obtained in saliva samples with different THC concentrations, using 67% Operon buffer+33% Saliva.

FIG. 18 illustrates three analysis methods that provide good precision as encompassed herein wherein dotted line represents the acceptable THC limit (5 ng/ml).

FIG. 19 illustrates binding curve using current at 1 min and binding curve using gain with currents at 1 min.

FIG. 20 illustrates antibody detection (here anti-NT-pro BNP antibodies) directly in bovine blood using the eSHRI assay and the specific epitope binding this antibody (ETSGLQEQRNHL).

### DETAILED DESCRIPTION

In accordance with the present description, there is provided an electrochemical steric hindrance hybridization assay for detecting analytes in a subject sample.

Remote detection of analytes in blood or saliva requires inexpensive, user-friendly sensors for molecular analysis in blood. Electrochemical DNA-based sensors (eDNA sensors) have shown great promise for the rapid detection of multiple classes of blood markers due to their high sensitivity, low cost, and relative insensitivity toward the matrix effects of biological samples. However, several challenges have been slowing down the commercialization of eDNA sensors. Among them, the performance of eDNA sensing mechanisms remains strongly affected by the variation of DNA probe density on the electrode surface.

Among the different signaling mechanisms developed for eDNA sensors, the recently developed steric hindrance hybridization assays have shown many advantages. Since the redox molecule is not located on the surface of the sensor when the sensor is first immersed in the biological sample, and DNA hybridization can take place in biological sample without interference, this specific eDNA sensing architecture works efficiently in whole blood without signal drift. Even though appearing relatively universal, this sensing mechanism, however, requires a high sensor density on the gold surface to achieve optimal performance. To solve this limitation, it is provided a novel electrochemical DNA hybridization sensing mechanism that employs multiple steric hindrance effects as well as a redox inhibition mechanism, which displays a higher gain as well as less dependence on the sensor density. For example, by strategically positioning the location of the recognition element on the signaling DNA, one could create a novel steric hindrance mechanism between the analyte and the gold electrode that, in principle, should remain insensitive to sensor density. Alternatively, bringing an analyte protein next to the redox molecule could also result in a contact-induced redox inhibition mechanism that would limit the electron transfer rate and the effect of probe density on the performance of the sensor.

Accordingly, in an embodiment, it is provided a novel electrochemical hybridization-based assay for detecting proteins that integrates four distinct signaling mechanisms to create a highly sensitive, density-independent sensing mechanism that performs reproducibly well even with fabrication variation or aging. This signaling mechanism uses protein analyte binding on a recognition element attached to a redox-active signaling DNA to inhibits its hybridization and redox activity on an electrode containing its complementary strand. Protein analyte binding in close proximity to the redox molecule may further limits electroactivity through a contact inhibition mechanism. As demonstrated herein, this novel sensing mechanism allows the detection of a low nanomolar concentration of protein analytes in a drop of blood in less than 3 mins, with nearly optimal signal gain (up to 93%).

As encompassed herein, and not limited to, the sample can be a biological sample such as whole blood, saliva, or urine, from a human or animal subject. It is also encompass that the sample can be water, environment element such as dirt or food.

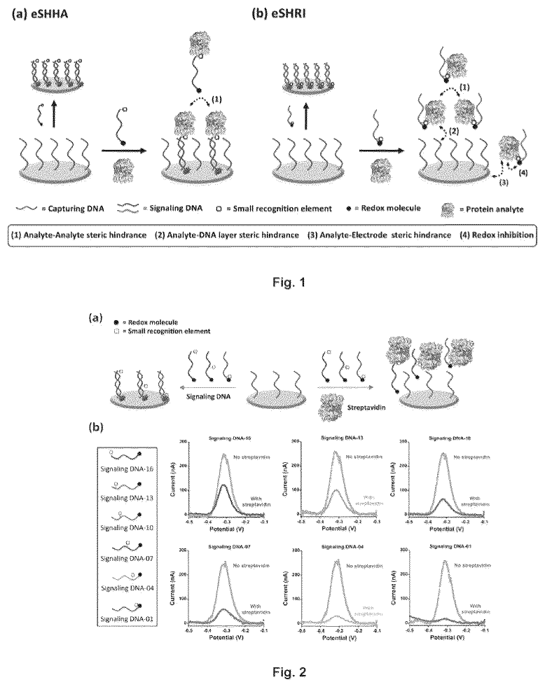

In the classic version of electrochemical steric hindrance hybridization assay (eSHHA, FIG. 1a), a signaling DNA containing a redox molecule and a recognition element at opposite extremity diffuses in the sample and hybridizes to a complementary capturing DNA located at the surface of the electrode. Upon protein analyte binding to the signaling DNA, fewer signaling DNA reach the electrode surface due to the steric hindrance produced between the large protein analytes (FIG. 1a). Unfortunately, eSHHA can only generate a high signal gain on the electrodes (not more than −60% signal reduction) with high surface density because the steric hindrance is dependent on the distance between the capturing DNAs on the surface. To create a density-independent sensing mechanism, a novel electrochemical steric hindrance and redox inhibition (eSHRI) hybridization assay is presented in FIG. 1b. The label position of the recognition element and the position where the analyte binds produces more steric hindrance. Analyte binding closer to the redox molecule results in additional steric hindrance between the protein analyte and the capturing DNA layer and even between the analyte and the gold surface. Alternatively, having the protein analyte binding in the vicinity of the redox molecule also leads to a contact-induced redox inhibition mechanism, a mechanism that could significantly reduce the electron transfer rate and improve the signaling mechanism of eDNA sensors.

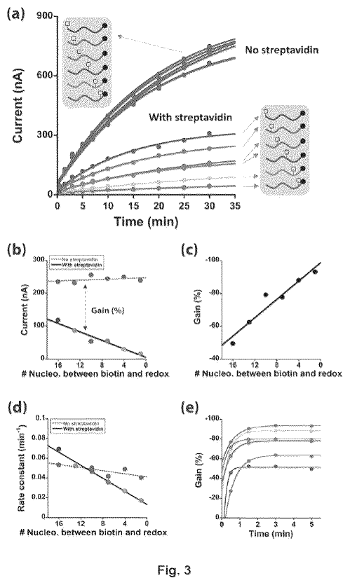

The hybridization efficiencies between a signaling DNA and a capturing DNA would vary with the position of the recognition element on the signaling DNA strand (FIG. 2a). To explore these novel steric hindrances and redox inhibition mechanisms, six signaling DNAs were designed that vary the location of the recognition element, here biotin, from the most distal location to the redox molecule (signaling DNA-16, FIG. 2b) to its most proximal location (signaling DNA-01, FIG. 2b). These 16 nucleotides long signaling DNAs display the same sequence and are all labeled with methylene blue at their 5′ extremity to generate electrochemical readout signal upon hybridizing to their complementary DNA attached on the gold surface. These DNAs were synthesized in-house using a DNA/RNA Synthesizer (K&A Laborgeraete, Germany), a methylene blue phosphoramidite to label the 5′ extremity and a modified thymine-biotin. In the absence of protein analyte, all signaling DNAs can hybridize on the electrode surface with similar efficiencies: all showed very similar electrochemical current at −0.3 V after 5 mins (FIG. 2b). This indicates that the location of the small biotin does not affect the efficiency of hybridization. In contrast, upon binding of the protein analyte (here streptavidin) to the signaling DNA, the efficiency of hybridization was primarily affected by the location of the recognition element. For example, the most distal location of biotin (signaling DNA-16) only reduced the hybridization efficiency by 50%, but the most proximal location reduced the hybridization efficiency by up to 93% after only 5 mins.

As used in the context of the present disclosure, the “target analyte” may be any molecule of interest which is suspected to be present in a sample to be analysed and is capable of binding (in an embodiment specifically) to the signaling DNA molecule. In the context of the present disclosure, the expression “specific binding” or “specifically bind” refers to the interaction between two elements in a manner that is determinative of the presence of the elements in the presence or absence of a heterogeneous population of molecules that may include nucleic acids, proteins, and other biological molecules. For example, under designated conditions, a target binds to a particular macromolecular entity and does not bind in a significant manner to other molecules in the sample. In some embodiments (when the target is considered to be a macromolecular entity), “specific binding” of the target to the signaling DNA molecule results in steric hindrance at the surface of the substrate which ultimately creates a detectable signal or a detectable change in a signal.

In an embodiment, the target analyte can have an average molecular weight of at least 10 kDa, at least 20 kDa, at least 30 kDa, at least 40 kDa, at least 50 kDa, at least 60 kDa, at least 70 kDa, at least 80 kDa, at least 90 kDa, at least 100 kDa, or at least >1000 kDa. In an embodiment, the target can be an antibody (IgA, IgE, IgG, IgM, IgD), a single-chain antibody or an antibody fragment (Fab′ fragment for example). The target can be a cell (such as, eukaryotic cell (e.g., an immune cell) or a prokaryotic cell (e.g., a bacteria)) or a cellular fragment (such as, for example, an erythrocyte or a platelet). The target can be an infectious agent, such as, for example, a prokatyotic cell (e.g., a bacteria), a fungal cell (e.g., a yeast or a mold), a virus or a prion. The target can also be a polymeric molecule (e.g., a polynucleotide molecule (DNA—(including cDNA and DNA fragments) and RNA-based (including mRNA, miRNA, tRNA, siRNA), a polypeptide molecule or a carbohydrate (e.g., a lectin) for example) or can be a monomeric molecule (e.g., a lipid for example). When the macromolecule is a polynucleotide molecule, it comprises at least 20, 30, 40, 50 or at least 100 nucleic acid bases. Exemplary polypeptides/proteins include, but are not limited to, antibodies, cellular receptors, secreted polypeptides, immuno-modulatory polypeptides (interleukins, interferons), hormones (such as growth factors), coagulation factors, DNA-binding polypeptides (such as transcription factors), etc. The target can be a single molecule, combination of two or more molecules (e.g., a glycosylated antibody for example) or an aggregation of two or more molecules. The target can be a naturally-occurring molecule or a synthetic (e.g., man-made) molecule.

The impact of protein binding location of the signaling DNA on the hybridization kinetics was further characterized. In the absence of streptavidin, all six signaling DNAs displayed very similar kinetic traces (FIG. 3a), the currents after 5 mins (FIG. 3b), and the hybridization rates (FIG. 3d). In contrast, upon streptavidin binding, all six signaling DNAs showed a decrease in hybridization efficiency inversely proportional to the distance between the biotin and the methylene blue (FIG. 3a). For example, moving the location of the biotin from the most distal location (signaling DNA-16) to the most proximal location next to the gold surface (signaling DNA-01) reduces hybridization by 86% after 5 mins (FIG. 3b) and the rate of hybridization by four folds (FIG. 3d). Interestingly, the signal gain of these streptavidin sensors increases linearly from −49% (signaling DNA-16) to −93% (signaling DNA-01) as the location of the biotin is moved closer to the gold surface or methylene blue (FIG. 3c). In addition to significantly improving the signal gain of the sensor, the provided assay is also very rapid with the maximal signal gain being reached after only two minutes (FIG. 3e).

A primary advantage of the eSHRI (electrochemical Steric Hindrance and Redox Inhibition hybridization assay) is that its performance should, in principle, be less dependent on the variation of the capturing DNA density on the electrode surface. The classic eSHHA mechanism is drastically affected by variation in capturing DNA densities: when the average distance between two capturing DNA becomes wider than the size of the protein analyte, there is no or few steric-hindrance. eSHHA, therefore, requires a high surface density to perform optimally (and is therefore sensitive to sensor degradation). In the eSHRI mechanism, it is expected that the analyte also creates steric hindrance with the DNA layer (which is also density-dependent) and with the electrode surface. As the protein analyte gets closer to the methylene blue, it is also possible that methylene blue binds the surface of the analyte thus resulting in redox signal inhibition, a potentially novel signaling mechanism in eDNA sensors. Analyte-electrode steric hindrance and the contact-induced redox inhibition mechanism should remain relatively insensitive to variation in capturing DNA densities.

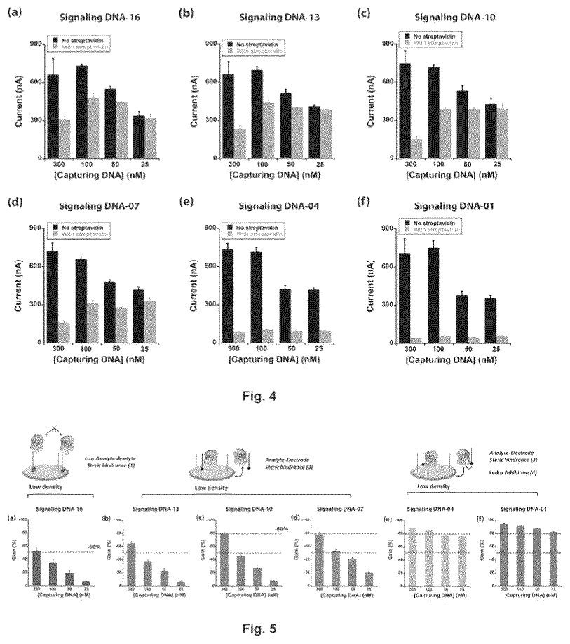

To verify this hypothesis, the streptavidin assay at different capturing DNA densities was investigated. As expected, the electrochemical currents were significantly reduced when decreasing the capturing DNA densities (FIG. 4). The efficiency of the steric hindrance assay was then assessed by testing these different sensors in the presence or absence of streptavidin. As expected, the signal gain for the classic eSHHA assay (signaling DNA-16) decreased from −53% to −7% upon decreasing the capturing DNA density (FIG. 5a). Moving the location of the biotin three nucleotides within the DNA layer (signaling DNA-13) created enhanced steric hindrance at a high surface density (−65% signal gain, FIG. 5b). This enhanced steric hindrance is likely occurring between the streptavidin and the DNA layer since this effect remains primarily dependent on the density of the capturing DNA. Deepening, even more, the location of biotin into the DNA layer (e.g., signaling DNA-10 and −07) further improved steric hindrance at a high surface density (−80% and −78% signal gain, FIGS. 5c-d) but less steric hindrance was also observed when decreasing the density of the DNA layer (−8% and −21% at low surface coverage, respectively, FIGS. 5c-d). In contrast, when moving the analyte binding site even closer to the electrode surface, for example signaling DNA-04 and -01, a large signal gain (−76% and −83%, FIGS. 5e-f) is also observed at a low density. These results suggest that the enhanced steric hindrance effect observed is no longer only due to (1) analyte-analyte steric hindrance or (2) analyte-DNA layer steric hindrance. The enhanced signal gain may be attributed to either a (3) analyte-electrode steric hindrance mechanism or due to a (4) redox inhibition mechanism between methylene blue and streptavidin. This novel sensing mechanism, which remains independent of the capturing DNA density offers a considerable advantage in terms of reproducibility since fabrication procedure and sensor aging typically modify DNA density and thus sensor performance.

The capturing DNA molecule is composed of any combination of known natural or synthetic nucleic acid bases and its backbone can be modified from naturally-occurring backbones. Naturally-occurring oligonucleotides contain phosphodiester bonds and synthetic oligonucleotides comprising nucleic acid analogs may have alternate backbones, comprising, for example, phosphoramide, phosphorothioate, phosphorodithioate, O-methylphosphoroamidite linkages and peptide nucleic acid backbones and linkages. Other analog nucleic acids analogs include those with positive backbones, non-ionic backbones, and non-ribose backbones. Nucleic acids bases containing one or more carbocyclic sugars are also included within the definition of contemplated nucleic acid bases.

To further explore the nature of the eSHRI mechanism, the length of the capturing DNA was varied to confirm the involvement of this later in creating the “analyte-DNA layer” steric hindrance. Reducing the length of the capturing DNA should display two effects: a) reduce the “analyte-analyte” steric hindrance since less signaling DNA can hybridize on the surface due to the lower affinity of the capturing DNA; b) reduce the “analyte-DNA layer” steric hindrance since the average distance between the analyte and the DNA layer is increased.

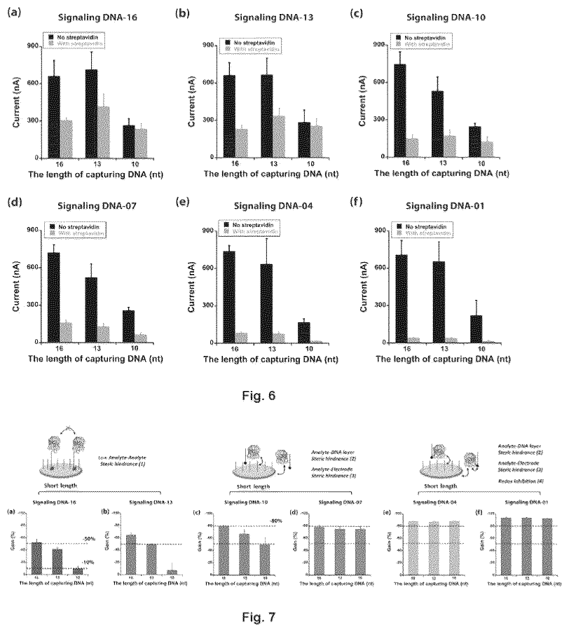

To investigate these effects, capturing DNAs were tested with decreasing length from 16, 13, to 10 nucleotides (nt). As expected, it was observed that the 10-nt capturing DNA greatly reduced the affinity for the signaling DNA (FIG. 6). Less signaling DNA can hybridize to the surface modified with the 10-nt capturing DNA, therefore drastically reducing analyte-analyte steric hindrance resulting in little signal gain upon adding streptavidin for signaling DNA-16 and -13 (FIG. 7a-b). Of note, when the biotin penetrates the DNA layer even farther with signaling DNA-10 and -07, the assay becomes less dependent on the number of signaling DNA on the surface: the low-affinity 10-nt capturing DNA still produces −50% (signaling DNA-10, FIG. 7c) and −75% (signaling DNA-07, FIG. 7d) signal gain upon adding streptavidin. These results confirm that when the analyte binds in the middle of the capturing DNA layer, the dominant steric hindrance effect is no longer the (1) analyte-analyte steric hindrance but rather either the (2) analyte-DNA layer steric hindrance or the (3) analyte-electrode steric hindrance. Finally, when inserting the biotin closer to the electrode with signaling DNA-04 and -01, the signal gain is further increased and remains constant with all capturing DNA lengths (FIG. 7e-f). These results suggest that additional steric hindrance with the gold electrode or a potential redox inhibition mechanism is obtained upon methylene blue binding to the surface of the protein analyte.

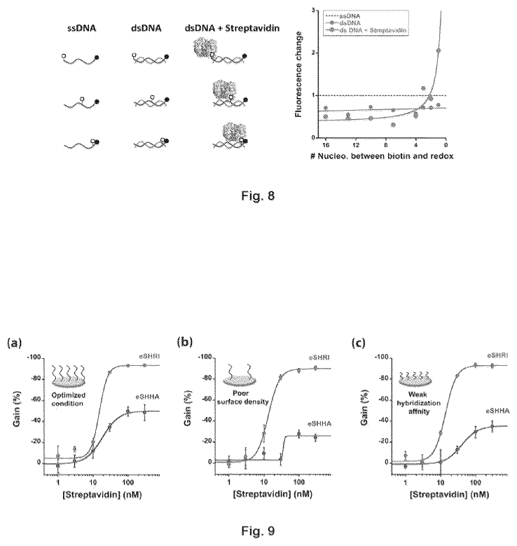

It is provided an electrochemical hybridization-based signaling mechanism for the detection of protein analytes that takes advantage of two novel steric hindrance mechanisms: analyte-DNA layer and analyte-electrode steric hindrance. This mechanism displays many advantages over previously developed signaling mechanisms (e.g., diffusion-collision and structure-switching) due to its insensitivity to variation in sensor density and nearly optimal signal gain (>−90%). However, a third effect (or mechanism) may also contribute to the overall performance of this new class of sensors: a redox inhibition mechanism between the methylene blue and the analyte protein may also limit electron transfer rate. To demonstrate the potential binding-induced redox inhibition mechanism observed when streptavidin binds next to methylene blue, the fluorescence of methylene blue was monitored following the addition of streptavidin binding at varying distances from it (FIG. 8). The fluorescence of the methylene blue-labelled signaling DNA is slightly reduced (˜32% on average) upon binding to its complementary strand (FIG. 8). This is a characteristic effect observed when the tumbling rate of a fluorescent-labelled molecule is decreased upon increasing its size. Streptavidin was then added to the various double-stranded signaling DNA and found that the fluorescence variation varied greatly depending on the distance between streptavidin and methylene blue (FIG. 8). When adding streptavidin far from the methylene blue (i.e., 16, 13, 10, 7, and 4 nucleotides separation), a 30% reduction of fluorescence intensity was observed. In contrast, when streptavidin binds near the methylene blue (less than three nucleotides apart), a large protein-induced fluorescence enhancement was noticed, which also correlates with a significant contact redox inhibition activity. These results, therefore, support the presence of physical contact between methylene blue and streptavidin, thus a novel electrochemical signaling mechanism based on contact-induced redox inhibition.

eSHRI displays higher signal gain and is drastically less sensitive to sensor fabrication parameters (e.g., surface density, length of capturing DNA) or to sensor degradation than classic eSHHA. To demonstrate whether eSHRI also reduces the detection limit and improves sensitivity, the dose-response curve obtained using the signaling DNA-16 (classic eSHHA) and signaling DNA-01 (eSHRI) were compared. At optimal conditions (i.e., at high surface density and long capturing DNA), both assays exhibit a sigmoid curve centered around the K_{D} of 19 nM and 15 nM (FIG. 9a), respectively. It is noted that classic eSHHA displays a 22% change of signal gain between 10 nM and 30 nM streptavidin concentration, while eSHRI reduces its signal gain by 66%, thus significantly improving sensitivity by 3-folds (FIG. 9a). As demonstrated above, the performance of eSHRI is also independent of various sensor fabrication parameters. For example, when surface density is reduced due to fabrication variation or sensor aging, classical eSHHA drastically reduces its performance, but eSHRI remains unchanged (Fig. b). Similarly, when hybridization efficiency is affected by factors reducing affinity between the signaling and capturing DNA (e.g., high temperature, destabilizing matrix/buffers), the performance of eSHRI remains unaffected while classic eSHHA is drastically affected (FIG. 9c). Overall, these results demonstrate that in addition to displaying an enhanced sensitivity, eSHRI also remains insensitive to sensor degradation and various exterior factors affecting sensor performance (batch to batch fabrication, temperature, and matrix).

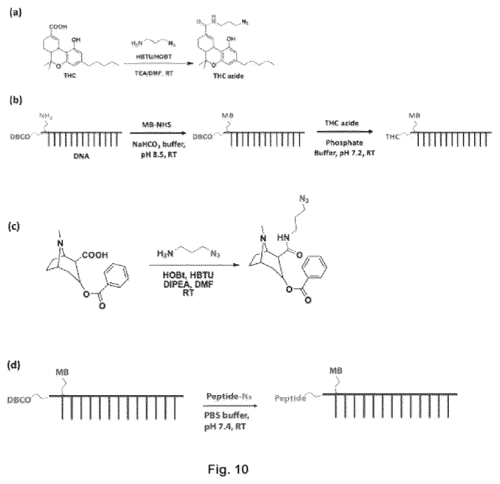

To demonstrate the versatility and potential universality of eSHRI, the assay was adapted for the detection of other more clinically relevant proteins, such as antibodies. As a proof-of concept, a model antigen was employed (or small recognition element), tetrahydrocannabinol (THC) to detect its specific antibody. A dual labeled signaling DNA was first designed on which methylene blue is attached, the redox molecule, and THC—the antigen (FIG. 10b). To do so, THC was first modified into THC azide for subsequent coupling reactions (FIG. 10a). Using a NH₂— and DBCO-labeled DNA obtained from IDT (Integrated DNA Technologies, Inc., Iowa, USA), methylene blue was first conjugated through an ester-amino reaction followed by attachment of THC-azide through azide-DBCO coupling reactions. In absence of THC antibodies, this dual labeled signaling DNA displayed efficient hybridization with current reaching over 300 nA after 10 min (FIG. 11). In contrast, in presence of 100 nM of THC antibody, the hybridization efficient decreases by over 60% even only after a few seconds (FIG. 11b). The fact that maximal signal decreased is reached even at the beginning of the hybridization reaction further indicates that the binding rate of THC to its antibody is much faster than the hybridization rate of the signaling DNA to its complementary electrode-bound DNA. Although impressive, is was also noted, however, that this 80% signal gain decrease is relatively smaller than the 93% signal decrease obtained for the streptavidin-biotin system (−93%). This could be due to the fact that one antibody is expected to bind two signaling DNA while Streptavidin could in principle bind 4 of them. Alternatively, it is possible that the anti-THC antibody, although in close proximity to methylene blue upon binding to THC may not be able to bind to methylene to further inhibits its redox activity (see e.g. FIG. 5f). The versatility of this signaling architecture was also further demonstrated by replacing THC attached on the signaling DNA with cocaine (FIG. 10c) or with a peptide epitope (FIG. 10d).

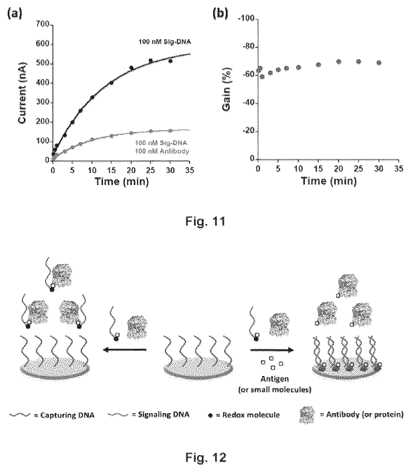

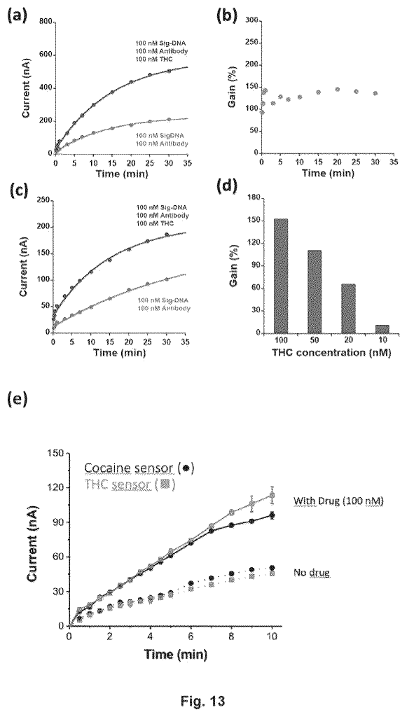

To broaden the scope of analytes detected by eSHRI, the usefulness of this assay was also demonstrated in a competition format for the detection of antigens or small molecule. As shown in FIG. 12, in the absence of antigen (or small molecules), a reagent antibody (or specific protein) is available to bind a signaling DNA that contains a copy of the antigen, resulting in a very low hybridization efficiency and thus low electrochemical signal due to the steric hindrances and redox inhibition. In the presence of antigen, the antibody preferentially binds this later instead of the antigen conjugated to the signaling DNA, thus leaving the signaling DNA unbound and free to efficiently hybridize to the capturing DNA on the surface of the electrode, generating a high electrochemical signal (FIG. 13a). As encompassed herein, the sample can be mixed with the antibody 0 to 1 min before adding the signaling DNA and adding the sample on the electrode to make sure the free antigen binds the antibody before the antibody binds the signaling DNA. Accordingly, it can result in an increase of the gain depending on the rate of the antigen-Ab interaction or the viscosity of the sample

The usefulness of this competition assay was further validated using the proof-of-concept THC antibody to detect free THC molecules (FIG. 13 a-d). In this experiment, 100 nM of signaling DNA, 100 nM THC antibody were employed. In the absence of free THC molecules, THC antibodies bind the THC-labeled signaling DNA, resulting in a low hybridization efficiency and current (FIG. 13a). In the presence of 100 nM of free THC molecules, the THC antibody binds this THC first (like in the control experiment, the antibody left reacted 5 min first with the sample before adding the signaling DNA) and the signaling DNA remains therefore unbound and reach the electrode surface in high efficiency (FIG. 13a). As encompassed herein, the incubation time can be as low as 30 sec, depending on the assay. Again, this competitive eSHRI assay also generates a nearly optimal gain in the first second of the hybridization (FIG. 13b). The ability of this competitive eSHRI to perform in complex biological media was also tested by detecting free THC molecules directly in human saliva (FIG. 13c). As shown in FIG. 13c, it was found that the performance of this assay did not significantly decrease when deployed directly in a biological sample. The overall raw current decreased in saliva may be attributable to some matrix effect of saliva that reduces electron rate transfer or hybridization efficiency. Finally, it is provided that this competition assay is quantitative and display detection limit near 10 nM (3.14 ng/ml) in saliva, which is within the detection limit required for commercial lateral flow THC detection kits in saliva (5 ng/ml). A similar assay for the detection of other small drugs, antigens or molecules can also be realized by simply replacing the THC with another molecule, and the THC-antibodies with the antibodies binding to this specific molecule. See for example the detection of cocaine using a cocaine-labeled DNA (FIG. 10c) and a cocaine antibody (FIG. 13e).

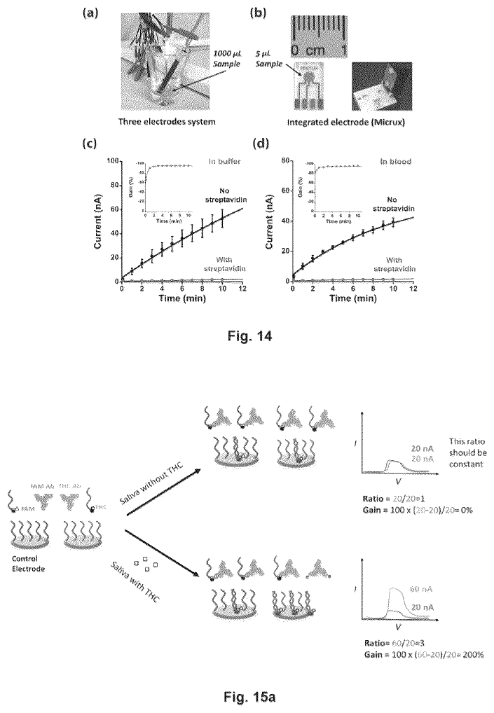

It is further demonstrated the ability of eSHRI to detect protein analytes directly in complex biological samples, such as whole blood using a single drop of blood (5 ul). To do so, streptavidin detection was tested directly in a drop of blood using a small integrated electrode. All results showed so far have been obtained using either a classic three electrodes system including a rod gold working electrode, a glass tubing Ag/AgCl reference electrode, and a platinum wire counter electrode, which typically requires large sample volumes of 1000 μL (FIG. 14a). Alternatively, CTI electrodes were also employed to demonstrate the detection of THC in saliva, which required 70 ul of sample. To test the sensor for point-of-care applications in a drop of blood, the assay was adapted on a smaller disposable electrode made using photolithography methods from Micrux Technologies. This Micrux electrode includes a gold working electrode (WE), a platinum reference electrode (RE), and a platinum counter electrode (CE) on a small glass chip (6*10 mm) (FIG. 14b). The smaller surface area of the electrode (0.785 mm²) only requires 5 μL sample volume (a small drop of blood) to perform the assay. Streptavidin was first detected in the buffer to compare the performance of both the Micrux and rod electrodes. It was found that both electrodes display similar high signal gain (Micrux=−96% at 5 mins, FIG. 14c inset; rod=−93% at 5 mins, FIG. 3c) and kinetics (Micrux=0.035 min⁻¹, FIG. 14c; rod=0.04 min⁻¹, FIG. 3d). The performance of these small streptavidin sensors was further tested directly in a drop of blood (FIG. 14d). It was found that the results in blood display comparable high signal gain (−95% at 5 mins) as in buffer but show slightly faster kinetics (0.1 min⁻¹). It is worth noting that the raw current in the blood is about 25% lower than that in buffer, which may be caused by the decrease of electron transfer rate in blood. These results demonstrate the high specificity and selectivity of eSHRI's signaling mechanism that remains insensitive to any nonspecific absorption of proteins on the surface of the sensor.

As encompassed herein, the substrate provides a surface for associating with at least two capturing DNA molecules to prevent them from freely diffusing in solution/suspension. In still another embodiment, the substrate is a metallic electrode (such as a gold, silver, platinum) or a non-metallic electrode (e.g., carbon or silicon for example). The conductive and semiconductive materials can be metallic or non-metallic.) and the reporter moiety is a redox reporter (e.g., organic redox moieties, such as viologen, anthraquinone, ethidium bromide, daunomycin, methylene blue, and their derivatives, organo-metallic redox moieties, such as ferrocene, ruthenium, bis-pyridine, tris-pyridine, bis-imidizole, and their derivatives, and biological redox moieties, such as cytochrome c, plastocyanin, and cytochrome c′).

In certain embodiments, the detection system described herein is capable of specifically identifying nanomolar or picomolar concentrations of targets in a sample. In some embodiments, the system has a dynamic range of at least 10, 20, 30, 40, 50, 60, 70 or 80. For example, in embodiments in which the signaling oligonucleotide is unimolecular and used at a concentration of 100 nM, the system can detect a target having a concentration ranging from 1 nM to 1 μM, such as from 1 nM to 750 nM, including from 1 nM to 500 nM, or from 1 nM to 250 nM, for instance from 1 nM to 100 nM or from 2 nM to 160 nM.

It is accordingly provided a density-independent electrochemical eDNA sensing mechanism that employs novel electrochemical steric hindrance and redox inhibition mechanisms (eSHRI). This new strategy aimed to solve many limitations of current eDNA sensors, including limited signal gain and high dependence on sensor fabrication and aging. Novel steric hindrance and redox inhibition mechanisms are created by decreasing the distance between the analyte binding site and the electrode. These mechanisms enhanced the signal gain up to −93% (close to the highest theoretical value of −100%) and rendered the signaling mechanism independent of sensor density on the surface of the electrode. Importantly, this new method achieves rapid (<3 mins) and one-step detection of protein analyte at low nanomolar range directly in a drop of blood.

Compared to the classical eSHHA that only uses one steric hindrance mechanism, eSHRI employs three different steric hindrance mechanisms: (1) analyte-analyte, (2) analyte-DNA layer, and (3) analyte-electrode surface. Furthermore, it is further described for the first time a (4) contact-induced redox inhibition mechanism that takes place when the protein analyte contacts and binds the redox molecule (methylene blue). eSHRI is a potentially universal signaling mechanism and maybe be adapted for the detection of other proteins analytes, such as clinically relevant antibodies (Covid-19 and HIV antibodies), by simply replacing the biotin with the specific antigen or epitope molecule (e.g. peptides). Alternatively, the assay could also be employed in a competition assay to detect any antigen. For example, it is provided how this assay could be easily adapted for the detection of THC directly in saliva.

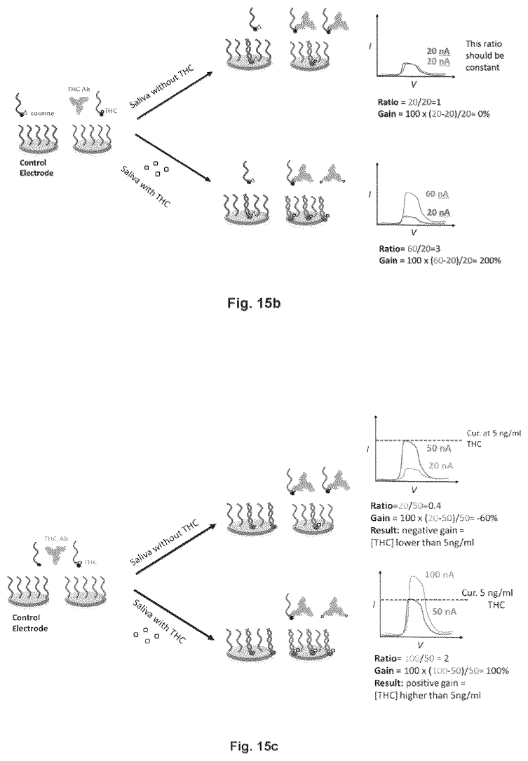

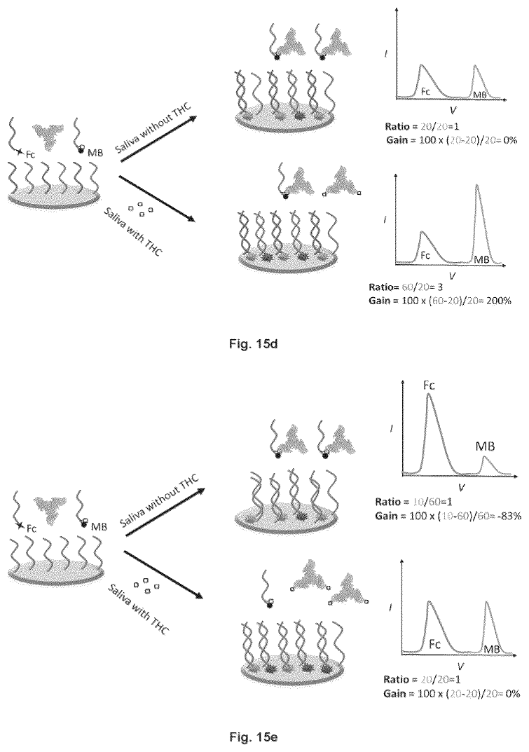

Accordingly, it is provided a mean for enabling rapid quantification of an analyte. Current measurements are typically report in gain, the % of current increase (or decrease) measured when a specific analyte (e.g. THC) is added to a sample. From the gain, it can be determine the concentration of analyte (e.g. [THC]) using a calibration curve, which reports gain versus [analyte]. In order to simplify the process of analyte quantification (e.g. THC), it is proposed five different strategies, which allow to determine the gain without requiring additional calibration steps in a sample that does not contain the analyte (FIG. 15).

Strategy 1: For an application that would require the detection of an analyte in a biological sample, a “sensor for detecting a rare non biological analyte” can be developed to detect a molecule absent from the clinical sample (FIG. 15a). This sensor will provide the require background current knowing that its analyte cannot be present in the clinical sample. For example, a Fluorescein sensor (FAM) that use FAM-labeled signaling DNA and a FAM antibody much similar to the THC sensor can be used to report on the stability of the signaling DNA, on the stability of the antibody and on the hybridization rate in this particular sample in a specific condition (for example different temperature).

Strategy 2: An other simple control could employs an extra electrode with another hybridization assay (different DNA sequences) that does contains another small molecule on the signaling strand (FIG. 15b). The concentration of this second signaling strand hybridizing a second electrode may be tune in such a way that its current represents the current obtained of the THC electrode in absence of free THC.

Strategy 3: An other simple control could employs an extra electrode with another hybridization assay (different DNA sequences) that does not contains THC on the signaling strand (FIG. 15c). The concentration of this second signaling strand hybridizing a second electrode may be tune in such a way that its current represents the “acceptable” legal concentration of THC (5 ng/ml) or of another analyte.

Strategy 4: An other simple control employs extra signaling and capturing DNA strands with different DNA sequence and redox element (e.g. ferrocene, which exchange electrode at an other voltage than methylene blue) that can also hybridize on the same electrode (the electrode is functionalized with the two different capturing DNA) (FIG. 15d). The hybridization of this second signaling strand can be tuned as required to create more or less signal (e.g. by varying the ratio of both capturing DNA) and is likely to remain relatively independent. Again the current generated by this control signaling DNA could be tune to represent a background current or the current typically obtained at [THC] (5 ng/ml).

Strategy 5: Represents a variation of the previous control electrode proposed. It employs an extra signaling DNA strands without recognition element but with a different redox element (e.g. ferrocene, which exchange electrode at an other voltage than methylene blue) that hybridize to the same capturing strand than the THC-signaling strand (FIG. 15e). The hybridization of this second signaling strand competes with the one of the THC-signaling. In absence of THC, this signaling DNA is expected to outcompete the THC-signaling bound to its Antibody. In presence of THC, this signaling DNA will hybridize at similar rate than the THC-signaling strand. This system will allow a ratiometric output with a likely stronger gain.

The present disclosure also provides kits and commercial packages comprising the detection, multiplex or control systems described herein. The kit can comprise any one of a reaction vessel (such as an electrode or an microtube-eppendorf) for each substrate that is being provided, a control sample known of lacking the target(s) intended to be detected, a control sample containing a known amount of the target(s) intended to be detected, a control value or a set of control values associated with the lack or the presence of known amounts of the target(s), solution or suspension comprising the substrate or the signaling DNA molecules as well as instructions on how to determine the presence/quantity of the target(s) based on the system that is provided. The instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-ray®, computer-readable memory, etc., on which the information has been recorded or stored. Yet another means that may be present is a website address which may be used via the Internet to access the information at a removed site. In an embodiment, the kit is an eppendorf wherein the signalling DNA molecules are added separately in the tube or in the cap of the microtube (FIG. 16). The eppendorf is then closed and shaken.

As illustrated in FIG. 16, the THC contained in a swab that had been used to brush someone's gum is released into a solution containing the THC antibody. Following a short incubation, the lid of the Eppendorf, which contains the dried signalling DNA (THC-DNA and control-DNA, here cocaine-DNA), is closed and the solution is mixed for 10 sec to solubilize the signalling DNA. This solution is then added to the electrodes (THC and cocaine electrodes) for analysis. As encompassed herein, the analysis can be performed on a portable potensiostat controlled by an app via Bluetooth. In absence of THC, both electrodes detect similar amount of signalling DNA. In presence of THC, less THC antibodies are available to bind to the THC-DNA and more THC-DNA can bind the THC electrode, leading to an increase in electrochemical signal on the THC electrode.

The methods described herein find use in a variety of different applications where determination of the presence or absence, and/or quantification of one or more targets in a sample is desired.

For example, the presence or absence or persistence of a target in a sample or significant changes in the concentration of a target over time can be used to diagnose/assess disease risk, presence of disease in an individual, or to tailor treatments for the disease in an individual. For example, the presence of a particular target or panel of targets may influence the choices of drug treatment or administration regimes given to an individual. In evaluating potential drug therapies, the presence, absence, or concentration of a target may be used as a surrogate for a natural endpoint such as survival or irreversible morbidity. If a treatment alters the target, which has a direct connection to improved health, the target can serve as a surrogate endpoint for evaluating the clinical benefit of a particular treatment or administration regime. Thus, personalized diagnosis and treatment based on the particular target or panel of targets detected in an individual are facilitated by the subject systems and methods. Furthermore, the early detection of targets associated with diseases is facilitated by the high sensitivity of the subject systems and methods, as described above. Due to the multiplex capability of detecting multiple targets in a single assay, combined with selectivity, sensitivity and ease of use, the presently disclosed systems and methods find use in quantitative, point-of-care or near-patient bio-molecular assays.

In a further example, the presence or absence of an infectious agent (such as a bacteria which can, in some embodiments, be resistant to one or more antibiotic) can be detected in biological samples, food or water.

In still another example, the presence or absence or persistence of a target in a sample or significant changes in the concentration of a target over time can be used to determine the contamination risk, presence of contamination in food.

The subject systems and methods find use in diagnostic assays, such as, but not limited to, the following: detecting and/or quantifying targets, as described above; screening assays, where samples are tested at regular intervals for asymptomatic subjects; prognostic assays, where the presence and or quantity of a target is used to predict a likely disease course; stratification assays, where a subject's response to different drug treatments can be predicted; efficacy assays, where the efficacy of a drug treatment is monitored; and the like.

The subject systems and methods also find use in validation assays. For example, validation assays may be used to validate or confirm that a potential disease biomarker is a reliable indicator of the presence or absence of a disease across a variety of individuals. The short assay times for the subject systems and methods may facilitate an increase in the throughput for screening a plurality of samples in a minimum amount of time.

In certain embodiments, the subject systems and methods find use in detecting antibodies in a sample. In some cases, the subject systems and methods may be used to detect the presence or absence of particular antibodies, as well as an increase or decrease in the concentration of particular antibodies in a sample.

In another embodiment, the subject systems can be used to screen for agents capable of modulating the binding between two biological entities. For example, the systems can be used to screen drug libraries and identify antagonist or agonists capable of increasing or lowering the binding between two biological entities.

### Example I

Tris (2-carboxyethyl) phosphine hydrochloride (TCEP) and 6-Mercaptohexanol (MCH) were purchased from Sigma Aldrich. Streptavidin was obtained from New England BioLabs. Whole blood (newborn calf) was purchased from Innovative Research. The columns and reagents for DNA synthesis were purchased from Biosearch Technologies and ChemGenes Corporation, respectively. The buffer used for the streptavidin is 50 mM NaH₂PO₄, 150 mM NaCl, pH 7.0. The buffer used for THC antibody or THC assay is 69.5 mM Boric acid, 254 mM NaCl, 18.8 mM Na₂HPO₄, 9.7 mM Chaps, 6.7 mM Proclin-150.

The DNAs were synthesized using a DNA/RNA synthesizer (K&A Laborgeraete, Germany). Unlabeled DNAs were purified by reverse-phase cartridge (RPC) while labeled DNAs (methylene blue-labeled or biotin-labeled) were purified using high-performance liquid chromatography (HPLC) equipped with a XBridge Oligonucleotide BEH C18 column (130 Å, 2.5 μm, 4.6 mm×50 mm, 1/pkg). The sequences of DNAs are listed in Table 1:

TABLE 1
Sequences of signaling DNA and capturing DNA

| Notes | Sequence (5′-3′) |
|---|---|
| Signaling DNA-16 | MB*-TCCT GCT CAT TCT CGT^{#} |
| | (SEQ ID NO: 1) |
| Signaling DNA-13 | MB-TCCT GCT CAT TCT CGT |
| | (SEQ ID NO: 2) |
| Signaling DNA-10 | MB-TCCT GCT CAT TCT CGT |
| | (SEQ ID NO: 3) |
| Signaling DNA-07 | MB-TCCT GCT CAT TCT CGT |
| | (SEQ ID NO: 4) |
| Signaling DNA-04 | MB-TCCT GCT CAT TCT CGT |
| | (SEQ ID NO: 5) |
| Signaling DNA-01 | MB-TCCT GCT CAT TCT CGT |
| | (SEQ ID NO: 6) |
| 16-nt Capturing DNA | ACG AGA ATG AGC AGGA-SH |
| | (SEQ ID NO: 7) |
| 13-nt Capturing DNA | AGA ATG AGC AGGA-SH |
| | (SEQ ID NO: 8) |
| 10-nt Capturing DNA | ATG AGC AGGA-SH |
| | (SEQ ID NO: 9) |

*MB = Methylene blue.
^{#}The underlined T in signaling DNA indicates the biotin
labeling.

The gold working electrodes (rod) (0.2 cm diameter, 0.0314 cm² surface area, West Lafayette, IN) were cleaned based on the literature (Xiao et al., 2007, Nat Protoc, 2:2875). The capturing DNA was immobilized on the clean gold electrode by the following procedures. Firstly, 1 μL of 100 μM capturing DNA and 2 μL of 10 mM TCEP was mixed for 1 hour at room temperature to reduce disulfide bond. Secondly, the reduced capturing DNA was diluted to the final concentration of 300 nM by buffer (50 mM NaH₂PO₄, 150 mM NaCl, pH 7.0), and then the gold electrode was incubated with the diluted capturing DNA solution (300 nM) for 2 hours at room temperature. Thirdly, the gold electrode was rinsed by deionized water to remove the non-immobilized capturing DNA on the surface, following further incubated with 2 mM MCH solution for 2 hours at room temperature to remove physically adsorbed capturing DNA and to passivate the gold electrode. Lastly, the functionalized gold electrodes were rinsed with deionized water for subsequent measurement or stored in buffer at 4° C. until use.

The electrochemical measurements were started immediately after putting capturing DNA functionalized gold electrode into the sample solution containing 100 nM signaling DNA and protein analytes. The electrochemical data was recorded by using square wave voltammetry (SWV) between −0.1 to −0.5 V. The peak currents were collected by using the manual fitting mode in the PSTrace 5.4 (2018) software. The kinetic profile of current versus time, signal gain versus time, and binding curves were fitted using Kaleidagraph, version 4.1 (2009). A EmStatMUX potentiostat multiplexer (Palmsens Instruments, Netherland) equipped with a standard three-electrodes cell containing a working electrode (gold rod electrode), a counter electrode (platinum, Sigma-Aldrich), and a reference electrode (Ag/AgCl (1 M KCl), CH Instruments) was employed to perform the electrochemical measurements at room temperature.

The Micrux electrodes (ED-SE1-AuPt, MicruX Technologies, Asturias, Spain) were fabricated by the following procedures. Firstly, the Micrux electrodes were cleaned by 0.05 M H₂SO₄ with cyclic voltammetry (−1.5 to +1.5 V with scan rate of 0.1 V/s, and the number of scans is 10). Secondly, the Micrux electrodes were functionalized with the capturing DNA using the same procedure as the rod gold working electrode. The electrochemical measurement on Micrux electrode was performed by adding 5 μL blood containing 100 nM signaling DNA and protein analytes to Micrux electrode surface. The experimental data was recorded by using square wave voltammetry (SWV) between −0.2 to −0.65 V. The peak currents were collected by using the manual fitting mode in the PSTrace 5.4 (2018) software.

The CTI electrodes (Conductive Technologies, York, PA, USA) were fabricated by the following procedures. Firstly, the CTI electrodes were cleaned by isopropanol for 2 mins. Secondly, the CTI electrodes were functionalized with the capturing DNA using the same procedure as the rod gold working electrode. The electrochemical measurement on CTI electrode were performed by adding 70 μL buffer or saliva containing 100 nM signaling DNA, 100 nM THC antibody, and THC analyte to CTI electrode surface. The experimental data were recorded by using square wave voltammetry (SWV) between −0.1 to −0.55 V. The peak currents were collected by using the manual fitting mode in the PSTrace 5.4 (2018) software.

The THC detection in saliva using the workflow provided in FIG. 16 with two electrodes was performed by:
1. Functionalizing two different capturing DNAs, namely the 16-nt Capturing DNA and 16-nt Capturing DNA-2, onto the two CTI electrodes.
2. Preparing an Eppendorf tube containing 28.5 nM of anti-THC antibody.
3. Preparing am eppendorf cap containing the two dried signaling DNA. Specifically, by simultaneously adding 2.5 μL of 10 μM THC-labeled signaling DNA and 1.35 μl of 5 μM Cocaine-labeled signaling DNA. The cap was allowed to dry in a dark place at room temperature for 2-3 hours.
4. Using a swab to collect THC from the gum for 30 sec. Then, by placing the swab into the Eppendorf tube and rotating it for 10 sec. Then removing the swab and letting the sample to incubate for 50 sec.
5. Finally, by covering the Eppendorf tube with the prepared cap containing the dried signaling DNAs. Mixing it upside down for 10-20 sec. Then adding three drops (or two if a lid is present on the surface of the electrode) of samples onto the two CTI electrodes for detection.

The sequences of capturing DNA and signaling DNA used were as follow.

16-nt Capturing DNA:
(SEQ ID NO: 7)
5′-ACG AGA ATG AGC AGGA-SH-3′
16-nt Capturing DNA-2:
(SEQ ID NO: 10)
5′-TGG ACA AAG AGG AGCA-SH-3′THC
labeled signaling DNA:
(SEQ ID NO: 11)
5′-MB-(THC) TCCT GCT CAT TCT CGT-3′
Cocaine-labeled signaling DNA:
(SEQ ID NO: 12)
5′-MB-(Cocaine) TGCT CCT CTT TGT CCA-3′
(here the cocaine-DNA was used as a control DNA
with the control protocol described in FIG. 15b).

Also provided is the electrochemical measurements for the antibody detection in whole blood. Electrochemical measurements were started immediately after mixing the signaling-DNA to the sample solution (whole bovine blood with or without antibodies), and applying it onto the gold electrode functionalized with the capturing DNA. The electrochemical data was recorded by using square wave voltammetry (SWV) between −0.10 to −0.55 V. The peak currents were collected by using the manual fitting mode in the PSTrace 5.9 (2022) software. The kinetic profile of current versus time, signal gain versus time, and binding curves were fitted using Kaleidagraph, version 4.1 (2009). A MultiEmStat3 (Palmsens Instruments, Netherland) equipped with a four-channels to perform the electrochemical measurements at room temperature.

The CTI electrodes (Conductive Technologies, York, PA, USA) were prepared as described previously ([0096]). The antibody was diluted to 10 uM with buffer. Original stock of signaling DNA of 100 uM was diluted to 10 uM with water. The electrochemical measurement on CTI electrode were performed by adding 70 μL whole bovine blood containing 100 nM signaling DNA and 60 nM antibody to the CTI electrode surface. The experimental data were recorded by using square wave voltammetry (SWV) between −0.10 to −0.55 V. The peak currents were collected by using the manual fitting mode in the PSTrace 5.9 (2022) software.

### Synthesis of THC-C9-Azide Linker:

To a stirred solution of (±)-11-nor-9-Carboxy-49-THC solution (2 mg) in DMF (500 mL), N,N,N′,N′-Tetramethyl-O-(1H-benzotriazol-1-yl) uronium hexafluorophosphate (HBTU) (2.3 mg) and 1-Hydroxybenzotriazole hydrate (HOBt) (0.8 mg) were added and allowed to stir for 10 min at room temperature. After 10 min, 3-Azido-1-propanamine (0.7 mg) and DIPEA were added and allowed to stir for overnight at room temperature. After completion of the reaction, the crude product was purified by HPLC using acetonitrile/water system (see FIG. 10a).

### Synthesis of Benzoylecgonine-Azide Linker:

To a stirred solution of benzoylecgonine (2 mg) in DMF (200 μL), N,N,N′,N′-Tetramethyl-O-(1H-benzotriazol-1-yl) uronium hexafluorophosphate (HBTU) (2.3 mg) and 1-Hydroxybenzotriazole hydrate (HOBt) (0.8 mg) were added and allowed to stir for 10 min at room temperature. After 10 min, 3-Azido-1-propanamine (0.7 mg) and DIPEA were added and allowed to stir for overnight at room temperature. After completion of the reaction, the crude product was purified by HPLC using acetonitrile/water system (see FIG. 10c).

### Synthesis of Methylene Blue Conjugated DNA:

DBCO containing amino-DNA (IDT-DNA) was dissolved in water. Methylene blue-NHS (20 mM) was added to IDT-DNA (2.6 mM) at the NH₂ of iAmMC6T 5′ end in sodium bicarbonate buffer solution (pH 8.5) and allowed to stir for overnight at room temperature. After completion of reaction, reaction mixture was purified by HPLC using acetonitrile and triethylamine (TEA)/acetic acid (TEAA) buffer solution. Methylene blue conjugated DNA (IDT-MB) was obtained in good yield (91%). See FIG. 10b.

### Synthesis of THC-C9 Conjugated IDT-MB:

IDT-MB (1.2 mM) in water, THC-C9-azide (2 mM) was added and allowed to stir for overnight at room temperature. After completion of reaction, reaction mixture was purified by HPLC using acetonitrile and triethylamine (TEA)/acetic acid (TEAA) buffer solution. THC-C9 conjugated IDT-MB DNA was obtained in good yields (90%). See FIG. 10b.

### Synthesis of Benzoylecgogine Conjugated IDT-MB:

IDT-MB (1.2 mM) in water, benzoylecgonine-azide (2 mM) was added and allowed to stir for overnight at room temperature. After completion of reaction, reaction mixture was purified by HPLC using acetonitrile and triethylamine (TEA)/acetic acid (TEAA) buffer solution. Benzoylecgogine conjugated IDT-MB DNA was obtained in appreciable yields (26%).

### Synthesis of Peptide Conjugated IDT-MB:

IDT-MB (0.2 mM) in water, peptide-azide (10 mg/mL) was added in 1 mM of PBS buffer (pH 7.4) and allowed to stir for overnight at room temperature. After completion of reaction, reaction mixture was purified by HPLC using acetonitrile and triethylamine (TEA)/acetic acid (TEAA) buffer solution. Peptide conjugated IDT-MB DNA was obtained in good yields (90%).

TABLE 1
Sequences of signaling DNA, capturing DNA and
peptide for antibody detection
using peptide epitope (FIG. 20)

| Notes | Sequence (5′-3′) |
|---|---|
| Signaling DNA-16 | 5DBCOTEG//iAmMC6T/CC TGC TCA |
| | TTC TCG T |
| | (SEQ ID NO 11) |
| 16-nt Capturing DNA | ACG AGA ATG AGC AGG A-SH |
| | (SEQ ID NO 13) |
| Peptide sequence | (N3)K GSGS ETSGLQEQRNHL |
| | (SEQ ID NO 14) |

*MB = Methylene blue. 5DBCOTEG = DBCO modification with
TEG linker. iAmMC6T = amino-modified thymine base.
(N3)K = Lysine amino acid with azide modification.

### Example II

### Example of Assay Optimization for THC Detection

### Optimizing the Dynamic Range of the Assay:

The dynamic range of the sensor can be tuned by changing the concentration of the antibody. An antibody binds 2 signaling DNA molecules so one need to make sure that there is always more antibody binding sites than signaling DNA molecules. A signaling concentration smaller than 2× the concentration of Ab is typically used to make sure the background remains small.

The sensitivity of the THC around the detection limit of THC (5 ng/ml or 15 nM) was optimized by employing 20 nM of antibodies and 35 nM of signaling DNA (see FIG. 16). Employing a higher concentration of antibody and signaling DNA would simply shift the dynamic range of the sensor towards high concentration of THC.

### Different Methods can be Employed to Quantify THC Concentration Via Current or Gain:

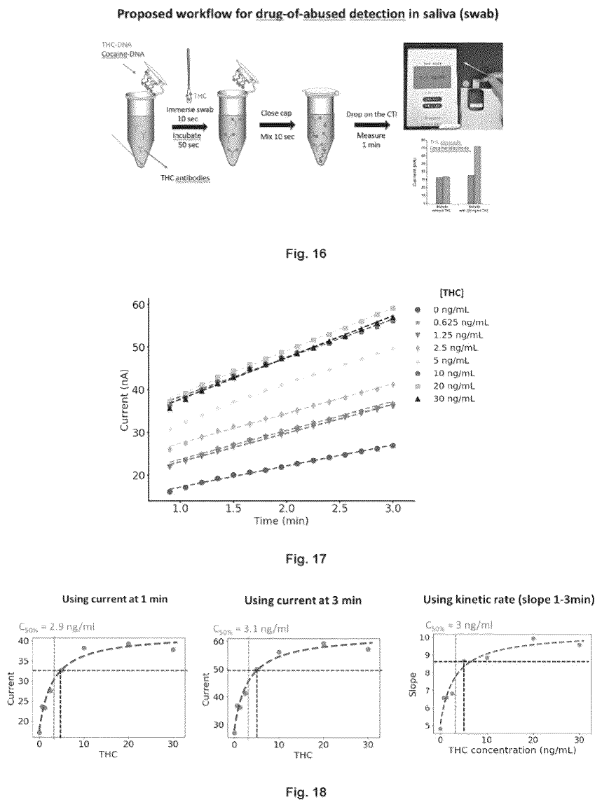

One method is to employ the current obtain after at a specific time (FIG. 16). It was found that the currents obtained before one minutes are less reproducible. FIG. 17 show binding curves obtained using the current at either 1 minute of 3 minutes after applying the sample on the electrode. For an assay that employs 20 nM of antibodies and 35 nM, the dynamic range observed is typically in the low nM (3-60 nM or 1-20 ng/ml). The current of the sensor (C_{S}) can easily be translated into a signal gain using the current of a control electrode (C_{C}) using:

$$Gain ( \% ) = 100 \times ( {C}_{S} - {C}_{C} ) / {C}_{C}$$

One other method is to employ the kinetics of hybridization (slope of the hybridization curve seen in FIG. 17). A slope between 1 and 3 minutes is typically employed and have observed that waiting longer time even increases the precision.

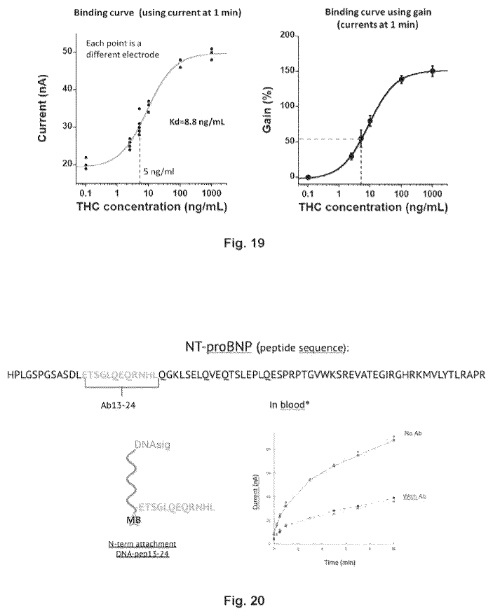

Example of a binding curve obtained for saliva samples containing different concentration of THC is provided (see FIG. 19). In these data the saliva sample was mixed ⅓ with a “THC-releasing buffer” that helps solubilize THC.

While the description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1: A system for detecting a target analyte in a sample, said system comprising:
a plurality of capturing DNA molecules having a first end and a second free end;
a first substrate having a surface associated with, at a plurality of locations, each of the first end of the plurality of capturing DNA molecules; and
a plurality of signaling DNA molecules, wherein each of the signaling DNA molecule:
has a core nucleic acid sequence which is substantially complementary to a region of each of the capturing DNA molecule and is capable of hybridizing with the capturing DNA molecule;
has a first end being associated with a moiety for binding an analyte entity in close proximity with a reporter moiety;
has a second end; and
is configured such that there is an inhibition of the hybridization of the plurality of signaling DNA molecules and reporter activity on the surface associated with the plurality of capturing DNA molecules upon binding of the moiety to the analyte;
wherein binding of the analyte to the signaling DNA molecule in proximity to the reporter moiety produces a steric hindrance between
(i) each analyte;
(ii) the analyte and the capturing DNA molecule; and
(iii) the analyte and the substrate.

2: The system of claim 1, wherein the moiety for binding the analyte entity is at a distance of at most 16 nucleotides form the reporter moiety.

3: The system of claim 1, wherein the moiety for binding the analyte entity is at a distance of at most 13 nucleotides, at most 10 nucleotides, at most 7 nucleotides, at most 4 nucleotides, or at most 1 nucleotide form the reporter moiety.

4: The system of claim 1, wherein the moiety is a at distance of at most 16 nucleotides, at most 13 nucleotides, at most 10 nucleotides, at most 7 nucleotides, at most 4 nucleotides, or at most 1 nucleotide from the first end of the signaling DNA molecule.

5: The system of claim 1, wherein the capturing DNA molecule is of at most 16 nucleotides in length, at most 13 nucleotides in length, at most 10 nucleotides in length, or at most 8 nucleotides in length.

6: The system of claim 1, wherein the target analyte is a macromolecule or an antibody.

7: The system of claim 1, for detecting a small molecule or a polypeptide, wherein the small molecule or the polypeptide competes for the binding of the analyte to the signaling DNA molecule.

8: The system of claim 7, wherein the small molecule is a drug substance.

9: The system of claim 8, wherein the drug substance is cocaine.

10: The system of claim 1, wherein the moiety for binding the analyte is an antigen.

11: The system of claim 1, wherein the reporter moiety is a redox-reporter or a fluorophore.

12: The system of claim 11, wherein the redox-reporter is methylene blue.

13: The system of claim 1, wherein the substrate is a metallic electrode, a 96-well plate, or a tube.

14. (canceled)

15: The system of claim 1, wherein the substrate is a glass tubing Ag/AgCl reference electrode, a platinum wire counter electrode, or a CTI electrode.

16: The system of claim 1, wherein the substrate is an electrode comprising a gold working electrode (WE), a platinum reference electrode (RE), and a platinum counter electrode (CE).

17: The system of claim 1, wherein the sample is a biological sample from a subject.

18: The system of claim 1, wherein the sample is water, an environment element or food.

19: The system of claim 17, wherein the biological sample is whole blood, saliva, or urine.

20. (canceled)

21: A kit comprising the system of claim 1 in a container.

22-24. (canceled)

25: A method for the detection of a target analyte in a sample, said method comprising:
providing the sample suspected of having the target analyte;
providing the system of claim 1;
providing or determining a control amount of the plurality of the capturing DNA molecules having hybridized with the plurality of the signaling DNA molecules in the system in the absence of the target analyte;
contacting the sample with the system;
determining a test amount of the plurality of capturing DNA molecules having hybridized with the plurality signaling DNA molecules in the system in the presence of the sample; and
characterizing the sample has having the target analyte if it is determined that the test amount is lower than the control amount and as lacking the target analyte if it is determined that the test amount is equal to or higher than the control amount.

26-32. (canceled)
